(19) United States
(12) Patent Application Publication
(10) Pub. No.: US 2010/0166831 A1
(43) Pub. Date: Jul. 1, 2010

(54) Microencapsulated Nucleotide Formulations for Aquaculture and Land Animal Feeds

(75) Inventors: Alfred J. Dentone, Acampo, CA (US); Robert A. Guthrie, London (CA); Delbert M. Gatlin, College Station, TX (US); Addision Lawrence, Port Aransas, TX (US); Peng Li, College Station, TX (US)

Correspondence Address: AMIN TALATI, LLC, 225 North Michigan Avenue, Suite 700, CHICAGO, IL, 60601, US

(73) Assignee: THE ROAN GROUP, INC, Acampo, CA (US)

(21) Appl. No.: 12/223,895

(22) PCT Filed: Feb. 12, 2007
(86) PCT No.: PCT/US07/03957

§ 371 Date: Feb. 5, 2009

Related U.S. Application Data
(60) Provisional application No. 60/772,303, filed on Feb. 10, 2006.

(51) Int. Cl. A61K 9/00; A61K 31/7088; A23K 1/16

(52) U.S. Cl. 424/442; 514/44 R

(57) ABSTRACT

A nucleotide-containing feed supplement having improved water stability and dietary efficacy is provided. The feed supplement includes a core containing at least one nucleotide microencapsulated within a coating such as a protein material. The microencapsulated nucleotide feed supplement can be combined with a nutrient base to provide a feed formulation adapted for use in aquatic environments. A method of making the microencapsulated nucleotide feed supplement is also provided.

This application claims the benefit of U.S. Provisional Application Ser. No. 60/772,303 filed on 10 Feb. 2006.

### FIELD OF THE INVENTION

This invention pertains generally to feed formulations and the bioavailability of nutrients to animals raised in aquaculture and, more particularly, to a microencapsulated nucleotide formulation that resists degradation in water and provides active nucleotides to the animal.

### BACKGROUND OF THE INVENTION

Aquaculture has played an increasingly significant role in the production of food as the world population continues to expand. The ability of the aquaculture industry to meet future production needs will depend on improved farming techniques and improved profitability to justify the investment in infrastructure. There is also an increasing need for intensive commercial farming of food fish, crustaceans, sport fish and other marine animals for restocking natural bodies of water to alleviate the pressures of over-fishing and/or the loss of natural habitat.

Commercial aquaculture can take the form of offshore cages or screened pens that take de of the natural tidal flow of water through the system to remove waste advantage and provide oxygenated water to support the animals in a protected, predator-free environment. Other forms of aquaculture take place inland in semi-closed or closed systems of tanks; ponds or raceways. Controlling the water temperature and oxygenation along with suitable natural and artificial feeds can assist to optimize growth conditions.

The juvenile and adult stages of many farmed aquatic animals can benefit from specialized feeds that support growth and aid in survivability and vitality of the particular type of animal. It will be seen that the controlled environment in aquaculture permits the introduction of additives to feeds that can stimulate growth, provide disease resistance and reduce mortality during development thereby maximizing yields and improving profitability. However, many potential feed supplements are water soluble or subject to degradation or Consumption by waterborne bacteria and the like and, therefore, do not have the intended effect on the cultured animals. There is a need for a low cost feed formulation that permits the efficient delivery of desired supplements to the digestive system of animals without significant losses.

### SUMMARY OF THE INVENTION

A general object of the invention is to provide a nucleotide-containing feed supplement having improved resistance to degradation or leaching in aquatic environments.

A more specific object of the invention is to overcome one or more of the problems described above.

The general object of the invention can be obtained, at least in part, through a feed supplement comprising a core including at least one nucleotide and a coating material, the nucleotide core encapsulated within the coating material. In accordance with certain embodiments, the core can further include an immune system stimulating compound and/or a growth stimulating compound. The feed supplement can have a water stability of from about 3 minutes to about 3 hours.

The prior art generally fails to provide nucleotide-containing feed supplements which exhibit enhanced water stability. Further, the prior art generally fails to provide a feed supplement including a nucleotide-containing core microencapsulated within a coating material which can be mixed or combined with a nutrient base or feed material to deliver desired supplements to the digestive system of aquatic animals with reduced loss of the supplements to the surrounding environment. Additionally, the prior art fails to provide microencapsulated nucleotide feed supplements having increased water insolubility and increased efficacy in enhancing growth and well being of cultured organisms.

The invention further comprehends a method of making a feed supplement which involves preparing a core mixture including at least one nucleotide and a thickening agent, preparing a coating material, combining the core mixture with the coating material to form a microencapsulated nucleotide material, drying the microencapsulated nucleotide material, and grinding the dried microencapsulated nucleotide material to form the feed supplement.

The invention still further comprehends a feed formulation including a nutrient base and a microparticulate nucleotide supplement. The microparticulate nucleotide supplement includes a core material encapsulated within a protein coating material. In accordance with certain embodiments, the core material can include one or more nucleotides and/or oligonucleotides alone or in combination with a thickening agent. The microparticulate nucleotide supplement can include about 20% to about 60% core material and about 30% to about 80% protein coating material.

The invention additionally comprehends a microencapsulated nucleotide supplement including about 1% to about 50% of at least one nucleotide, about 1% to about 30% of a thickening agent, and about 30% to about 70% of a protein coating material. In accordance with certain embodiments, the microencapsulated nucleotide supplement can further include about 0.01% to about 50% of an immune system stimulating compound and/or a growth stimulating compound.

As used herein the term “microencapsulated” refers to a particle or droplet of material surrounded by a coating to give or form small capsules or microcapsules. The material inside of a microcapsule can be referred to as a core, internal phase or fill. The coating surrounding the particle or droplet can be alternatively referred to as a wall, shell or membrane. The shape and surface, texture of the microcapsules can be smooth and/or spherical or, alternatively, can be rough and/or irregular.

Other objects and advantages will be apparent to those skilled in the art from the following detailed description taken in conjunction with the examples and the appended claims.

### DETAILED DESCRIPTION

The invention provides a nucleotide-containing feed supplement having improved resistance to degradation and/or leaching in aquatic environments. Such feed supplements typically include at least one nucleotide microencapsulated within a coating material.

Nucleotides are the building blocks of tissue RNA, DNA and ATP used in fast multiplying cells during growth and/of in an immune response. According to one aspect of the invention; an aquaculture feed formulation is provided that is supplemented with nucleotides for RNA, DNA and combinations thereof. The nucleotides can be provided alone or in combination with amino acids proteins and other essential nutrients or compounds that have been shown to stimulate growth and/or provide disease resistance.

Dietary nucleotides have been shown to improve feed uptake by some animals and enhance the morphology of the intestinal tract resulting in improved utilization of the feed. In some animals, dietary nucleotides can significantly improve disease resistance by supporting the immune system of the animal. One limitation to the use of nucleotides as a dietary supplement in aquaculture feeds is the ability to deliver sufficient quantities of biologically active nucleotides to the digestive system to be absorbed and used by the animal.

In accordance with the invention, a feed supplement or microencapsulated nucleotide supplement includes a core including at least one nucleotide and a coating, the nucleotide core microencapsulated within the coating. Advantageously, the core includes a nucleotide preparation comprising select, purified nucleotides alone or in combination with other proteins, amino acids, nucleotide precursors, medicines and/or other beneficial compounds. Suitable nucleotides include monophosphate nucleotides, such as, for example, adenosine monophosphate (AMP), cytidine monophosphate (CMP), uridine monophosphate (UMP), guanosine monophosphate (GMP), inosine 5′-monophosphate (IMP), uracil; oligonucleotides, and combinations thereof. In general, the core can include about 10% to about 60% by weight of at least one nucleotide. In accordance with certain embodiments, the core can include about 30% to about 60% by weight of at least one nucleotide or about 40% to about 50% by weight of at least one nucleotide.

The core can additionally include an immune system stimulating or immunopotentiating compound. In general, the core can include an immune system stimulating compound selected from beta-glucans, chitin, peptidoglycans, saponins and combinations thereof. Other immunopotentiating compounds which can be used in the core include various prebiotic and/or probiotic compounds whose effectiveness may be enhanced by microencapsulation.

In accordance with certain embodiments, the core can include about 0.01% to about 50% by weight of an immune system stimulating compound, or about 0.01% to about 25% by weight of an immune system stimulating compound or about 0.01% to about 2.5% by weight of an immune system stimulating compound. For example, in accordance with one embodiment, the core can include about 0.01% to about 2.5% by weight beta-glucans.

Some growth stimulating or supporting compounds such as, for example, amino acids and Many vitamins, may not be available for use in feed formulations dispersed in water because of the inability to deliver sufficient or efficacy quantities of such compounds to the animals. However, it has been discovered that such biologically active compounds can be effectively delivered through microencapsulation.

Accordingly, the core can further include growth stimulating or supporting compounds such as, for example; amino acids, hormones, and/or pharmaceutical compositions or drugs such as, for example, levamisole. Other biologically active compounds such as, for example, prebiotic, probiotic and/or other water soluble compounds which would benefit from microencapsulation can also be included in the core.

In accordance with certain embodiments, the core can include about 0.01% to about 60% by weight of a growth stimulating compound or about 1% to about 60% by weight of a growth stimulating compound or about 2.5% to about 50% by weight of a growth stimulating compound

The core can be in the form of a solid material such as, for example, a particulate or crystalline material, a liquid, an emulsion, a suspension of solids, and/or a suspension of smaller microcapsules. A thickening agent can be added to or combined with the at least one nucleotide to increase the viscosity of the core material and enhance or assist the microencapsulation process. Suitable thickening agents include, but are not limited to, cellulose derivatives such as, for example, carboxymethyl cellulose (CMC). In accordance with certain embodiments, the core can include about 30% to about 90% by weight thickening agent or about 30% to about 60% by weight thickening agent.

The core is advantageously surrounded or microencapsulated by a coating material which limits leaching of components from the core of the feed supplement into the water when fed to aquatic animals. Utilizing such a coating material to encapsulate and protect the core components can result in growth enhancement of fish, shrimp and/or other crustacean species when fed a feed supplement including a nucleotide preparation as described above.

In practice, any material that will provide a barrier to water for the core material but will degrade in the digestive system and release its contents after consumption by an animal can be used as a coating material. It will be understood that the coating materials can be varied to increase or decrease the half life of the coating within water as well as degradation with the digestive system of the animal.

Advantageously, the coating material can be selected to impart a desired level of water stability to the feed supplement. For example, the coating material may be selected to provide a feed supplement having a water stability of at least several minutes or about 3 minutes to about 3 hours or about 1 hour to about 3 hours. As used herein, the term “water stability” refers to the length of time during which the coating material of a microencapsulated nucleotide supplement remains intact and/or reduces leaching or dissolution of the core material into a surrounding aquatic environment.

Suitable coating materials include, but are not limited to, animal and/or vegetable proteins such as, for example, caseinate, soy concentrate, soy isolate, soy flour, wheat gluten, egg albumin, milk albumin, gelatin, zein (corn protein), whey protein concentrate and combinations thereof. In accordance with certain embodiments, the coating material can include a protein material such as, for example, casein, gelatin, whey protein concentrate and combinations thereof.

In general, the feed supplement or microencapsulated nucleotide supplement can include about 1% to about 60% by weight of at least one nucleotide and about 30% to about 99% by weight of a coating material such as, for example, a protein coating material. For example, the teed or microencapsulated nucleotide supplement can include about 20% to about 60% by weight of at least one nucleotide and about 30 to about 80% by weight of a coating material. In accordance with certain other embodiments, the feed or microencapsulated nucleotide supplement can include about 35% to about 60% by weight of at least one nucleotide and about 40% to about 65% by weight of a coating material. In accordance with certain additional embodiments, the feed supplement can also include about 1% to about 30% by weight of a thickening agent. For example, in accordance with one embodiment, the feed or microencapsulated nucleotide supplement can include about 1% to'about 50% by weight of at least one nucleotide, about 1% to about 30% by weight of a thickening agent and about 30% to about 65% by weight of a protein coating material. Such feed supplement can further include about 0.01% to about 50% by weight of an immune system stimulating compound and/or a growth stimulating compound.

The feed supplement can be prepared in a variety of particle sizes. Suitably, the particle size can be adjusted depending on the size of an associated diet for a target organism. For example, the particle size of the feed supplement can be adjusted to accommodate feeding of fish or crustaceans at various stages of development (e.g., for larval or juvenile aquatic organisms). Advantageously, the size of the feed supplement is selected such that it will not interfere with the taste and/or texture of an associated feed formulation so that the feed formulation will be attractive to fry and, therefore, consumed entirely.

In practice, the feed supplement can have a particle size in a range of about 20 microns to about 150 microns. In accordance with certain embodiments, the feed supplement can have a particle size in a range of about 20 microns to about 100 microns. In accordance with certain other embodiments, the feed supplement can have a particle size in a range of about 40 microns to about 100 microns in diameter.

In practice, the feed supplement can be included in or added to traditional feed formulations or diets such as, for example, an aquaculture feed formulations. In one embodiment, a feed formulation can include a nutrient base and a microparticulate nucleotide supplement including a cote material encapsulated within a protein coating material. Suitably, the core material includes at least one nucleotide and/or oligonucleotide alone or in combination with a thickening agent, an immune system stimulating compound and/or a growth stimulating compound. In accordance with certain embodiments, the microparticulate nucleotide supplement can include about 20% to about 50% by weight core material and about 40% to about 65% by weight protein coating material. Suitably, the feed formulation is produced with a density of less than about 1 gram/milliliter such that the feed can float in water.

A method for making the feed formulation involves combining or mixing the microparticulate nucleotide supplement with a nutrient base. Suitably, the nutrient base can be adapted for the physiology of unique aquatic animals. Advantageously, the microparticulate nucleotide supplement can be mixed or combined with a mineral and/or vitamin supplement to form a premix and the premix can be mixed into the nutrient base.

It will be understood that the encapsulation or coating process used to prepare feed supplement of the present invention can be varied to increase or decrease the half life of the coating in water as well as degradation within the digestive system of the animal.

A method of making a feed, microparticulate nucleotide, or microencapsulated nucleotide supplement in accordance with the invention involves preparing a core mixture including at least one nucleotide, preparing a coating material, combining the core mixture with the coating material to form a microencapsulated nucleotide material, drying the microencapsulated nucleotide material, and grinding the microencapsulated nucleotide material to form the feed supplement. In practice, the microencapsulated nucleotide material may be freeze-dried or spray-dried prior to grinding.

Alternatively, the feed supplement can be made using a spray-drying method. An advantage of preparing the feed supplement in this manner is the ability to handle labile materials because of the short contact time in the dryer. In certain embodiments, a method of making the feed supplement involves dissolving or suspending the nucleotide core materials in a melt or solution of the coating material and spray-drying the core/coating mixture such that core materials become trapped or encapsulated within the dried particles.

The present invention is described in further detail in connection with the following examples which illustrate or simulate various aspects involved in the practice of the invention. It is to be understood that all changes that come within the spirit of the invention are desired to be protected and, thus, the invention is not considered to be limited by these examples.

### EXAMPLES

As shown in TABLES 1-3, below, laboratory feeding trials have provided consistent positive results for one finfish species (red drum, Sciaenops ocellatus) and one crustacean species (Pacific white shrimp Litopenaeus vannamei) which constitute important aquaculture species around the world.

Feed Supplement Preparation:

By way of example and not of limitation, a microencapsulated nucleotide supplement was prepared according to the following scheme:
1. 2 grams each of AMP, CMP, UMP, GMP and IMP were measured and mixed together to form a nucleotide premix.
2. 10 grams of the nucleotide premix was dissolved in 500 ml of water.
3. 25 grams of CMC was added to the water/nucleotide mixture and mixed until it formed a dough-like consistency.
4. A coating was prepared by adding 35 grams of gelatin into 500 ml of hot water and mixing thoroughly. 30 grams of casein was then added to the gelatin mixture.
5. The hot casein-gelatin mixture was poured into the nucleotide/CMC mixture and mixed thoroughly.
6. The mixture from step 5 was placed in an ultra cold freezer for one hour, then placed into freeze-dry jars and freeze-dried overnight.
7. The freeze-dried product was ground and sieved.

The resulting microencapsulated nucleotide supplement contained about 2% AMP sodium, 2% CMP sodium, 2% GMP sodium, 2% UMP sodium, 2% IMP sodium, 25% CMC, 30% casein and 35% gelatin by weight.

Although a casein-gelatin coating is used in the preparation of the feed supplement, it will be understood that other coating materials may be used that can provide a barrier to water for the core materials, but will degrade in the digestive system and release its contents after consumption by the animal.

### Example 1

### Juvenile Litopenaeus vannamei

A growth trial was conduced at high stocking density in indoor tanks to evaluate nucleotide feed supplements in a semi-purified base diet. In the growth trial, diets were compared in terms of growth and survival of juvenile Litopenaeus vannamei (Pacific white shrimp) after five weeks. High rates of water recirculation and exchange, i.e., 12 gallons per hour (GPH) and 2 gallons per minute (GPM), respectively, and aeration were used to insure that water quality was not limiting growth and survival. Natural feeds were not available in the culture system.

Two test diets and two base diets were evaluated in the study. The two base diets include 25% and 35% protein, respectively. The test diets were prepared by adding 0.4% of the microencapsulated nucleotide feed supplement described above to a base diet.

Shrimp fed the base diet and the test diet including microencapsulated nucleotide feed supplements were stocked at a density of 100 shrimp/m². Results of the study are presented in TABLE 1.

TABLE 1

| | Initial weight | | |
|---|---|---|---|
| | (g) | Final weight (g) | Survival (%) |
| Dietary Protein (%)/ | | | |
| Nucleotide (%) | | | |
| 25/0 | 0.83 ± 0.06 | 10.44 ± 1.00 | 97.5 ± 7.9 |
| 35/0 | 0.84 ± 0.08 | 11.32 ± 1.32 | 90. ± 24.2 |
| 25/0.4 | 0.84 ± 0.04 | 10.96 ± 1.08 | 97.5 ± 7.9 |
| 35/0.4 | 0.88 ± 0.04 | 12.32 ± 1.31 | 95 ± 10.5 |
| ANOVA Pr > F | | | |
| Protein | 0.314 | 0.005 | 0.277 |
| Nucleotide | 0.215 | 0.051 | 0.584 |
| Protein × Nucleotide | 0.409 | 0.531 | 0.584 |

Values represent means of ten replicates ± SD

As shown in TABLE 1, juvenile shrimp fed an aquaculture diet supplemented with the microencapsulated nucleotides supplement described above demonstrated increased weight gain and survivability as compared to juvenile shrimp fed a standard diet unsupplemented base diet.

### Example 2

### Juvenile Sciaenops ocellatus

A study was conducted to determine if, juvenile Sciaenops ocellatus fed a base diet supplement with the microencapsulated nucleotide feed supplement described above would demonstrate increased weight gain, improved efficiency in nutrient uptake from feed and improved survival rates.

The growth and survival performance of juvenile Sciaenops ocellatus (red drum fish) fed isonitrogenous basal diets containing about 30% to about 40% protein, with and without the microencapsulated nucleotide feed supplement described above, for 4 weeks was assessed: The results of this study are presented in TABLE 2.

TABLE 2

| Diet | Weight gain (g) | Feed efficiency | Survival (%) |
|---|---|---|---|
| Basal | 187.0 ± 5.9 | 0.87 ± 0.03 | 95.83 ± 1.86 |
| Basal + Nucleotides | 203.7 ± 3.0 | 0.93 ± 0.01 | 98.9 ± 1.1 |
| ANOVA Pr > F | 0.011 | 0.012 | 0.162 |

As shown in TABLE 2, juvenile Red Drum fish feed a basal diet supplement with microencapsulated nucleotides demonstrated increased weight gain, improved feed efficiency and enhanced survival rates compared to juvenile Red Drum fish fed a standard, unsupplemented diet.

### Example 3

### Juvenile Litopenaeus vannamei

A growth trial was conducted at high stocking density in indoor tanks to evaluate nucleotide feed additives in a semi-purified base diet containing 35% protein. In the growth trial, diets were compared in terms of growth and survival of juvenile Litopenaeus vannamei after 42 days. At the conclusion of the trial, animals were evaluated with respect to immune response. Aeration and high rates of water recirculation and exchange were used to insure that water quality was not limiting to growth and survival. Natural feeds Were not available in the culture system.

Shrimp fed the nucleotide additives were stocked at a density of 100 shrimp/m². To document the effects of stocking at high density, a control treatment was stocked at a density of 10 shrimp/m² and fed the base diet without any nucleotide additives.

Two additional treatments, stocked at both 10 and 100 shrimp/m², were fed a high quality commercial aquaculture diet, Rangen 45/10 available from Rangen, Inc., Buhl, Id., USA., as a control to confirm that the semi-purified diet was adequate for good growth and survival.

A total of ten diets, as shown in TABLE 3, were prepared and/or fed to juvenile Litopenaeus vannamei. Experimental diets were prepared by adding encapsulated or nonencapsulated nucleotides to a semi-purified base diet containing 35% protein. The test groups were fed 15 times daily with an automatic feeder at a rate of 2.5 g/shrimp per week.

TABLE 3

| | | % | Density |
|---|---|---|---|
| Label | Diet | Nucleotide | (Shrimp/m²) |
| EPG.1 | Peng-Gatlin encapsulated pure | 0.1 | 100 |
| ERA.1 | ROAN encapsulated practical A | 0.1 | 100 |
| ERA.4 | ROAN encapsulated practical A | 0.4 | 100 |
| ERB.1 | ROAN encapsulated practical B | 0.1 | 100 |
| ERB.4 | ROAN encapsulated practical B | 0.4 | 100 |
| ERC.1 | ROAN encapsulated practical C | 0.1 | 100 |
| ERC.4 | ROAN encapsulated practical C | 0.4 | 100 |
| NER.1 | ROAN nonencapsulated practical | 0.1 | 100 |
| NER.4 | ROAN nonencapsulated practical | 0.4 | 100 |
| B*1 | Base diet - 35% protein | 0.0 | 10 |
| B*10 | Base diet - 35% protein | 0.0 | 100 |
| R*1 | Rangen 45/10 | 0.0 | 10 |
| R*10 | Rangen 45/10 | 0.0 | 100 |

The Peng-Gatlin encapsulated diet (EPG.1) included 0.1% of the microencapsulated feed supplement described above added to a 35% protein semi-purified base diet. The ROAN encapsulated diets (ERA.1, ERA.4, ERB.1, ERB.4, ERC.1, and ERC.4) included at least about 1% to a maximum of at least one nucleotide as previously described. The results of the 42 day growth study are shown in TABLE 4.

TABLE 4

| Diet | % Survival | Weight gain (g) | Growth rate (g/week) |
|---|---|---|---|
| EPG.1 | 82.50 ± 17.08 | 8.76 ± 1.62 | 1.43 ± 0.27 |
| ERA.1 | 70.00 ± 20.00 | 9.71 ± 1.09 | 1.62 ± 0.18 |
| ERA.4 | 93.33 ± 5.16 | 9.74 ± 1.19 | 1.62 ± 0.20 |
| ERB.1 | 95.00 ± 8.37 | 9.36 ± 1.51 | 1.56 ± 0.25 |
| ERB.4 | 90.00 ± 8.94 | 8.09 ± 2.08 | 1.35 ± 0.35 |
| ERC.1 | 90.00 ± 12.65 | 8.76 ± 1.12 | 1.46 ± 0.19 |
| ERC.4 | 85.00 ± 10.49 | 9.02 ± 0.80 | 1.50 ± 0.13 |
| NER.1 | 78.33 ± 7.53 | 9.21 ± 1.04 | 1.54 ± 0.17 |
| NER.4 | 86.67 ± 5.16 | 9.18 ± 0.92 | 1.53 ± 0.15 |
| B*1 | 100.00 ± 0.00 | 11.07 ± 1.42 | 1.84 ± 0.24 |
| B*10 | 83.33 ± 5.16 | 8.84 ± 1.76 | 1.47 ± 0.29 |
| R*1 | 100.00 ± 0.00 | 9.79 ± 2.18 | 1.63 ± 0.36 |
| R*10 | 90.00 ± 10.95 | 6.31 ± 1.17 | 1.05 ± 0.20 |

As shown in TABLE 4, this study demonstrates that diets including the ROAN encapsulated nucleotides aid in increased survivability, assist in greater weight gain, and further assist in better uptake of feed to increase the growth rate in high stocking density situations.

The microencapsulation of nucleotides and other water soluble constituents are believed to have the following benefits:
1. The protection conferred by such a coating or microencapsulation process increases the efficacy of nucleotide preparations.
2. The components used in the microencapsulation process as well as in the nucleotide preparation are natural products that are generally recognized as safe.
3. The components of the coating material will not accumulate in the animal.

Accordingly, coating or microencapsulation of nucleotide preparations or other water-soluble dietary components using the above describe materials and/or methods can reduce or prevent leaching of such dietary components in water and, thus, increase the efficacy of such dietary components as reflected in improved weight gain, feed efficiency and health. Additionally, it is believed that there are no known disadvantages or limitations to using the above described materials and/or methods to prepare nucleotide-containing feed supplements.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

We claim:

1. A feed supplement, comprising:
a core including at least one nucleotide; and
a coating, the nucleotide core microencapsulated within the coating.

2. The feed supplement of claim 1, wherein the at least one nucleotide is selected from the group consisting of AMP, CMP, UMP, GMP, IMP and combinations thereof.

3. The feed supplement of claim 1, wherein the core further comprises an immune system stimulating compound selected from the group consisting of beta-glucans, chitin, peptidoglycans, saponins and combinations thereof.

4. The feed supplement of claim 1, wherein the core further comprises a compound selected from the group consisting of uracil, oligonucleotides, nucleotide precursors and combinations thereof.

5. The feed supplement of claim 1, the further comprises a growth stimulating compound selected from the group consisting of amino acids, hormones, vitamins, minerals and combinations thereof.

6. The feed supplement of claim 1, wherein the coating comprises a protein material selected from the group consisting of casein, gelatin, whey protein concentrate and combinations thereof.

7. The feed supplement of claim 1, wherein feed supplement has a water stability of about 3 minutes to about 3 hours.

8. A method of making the feed supplement of claim 1, comprising:
preparing a core mixture including at least one nucleotide and at least one thickening agent;
preparing a coating material;
combining the core mixture with the coating material to form a microencapsulated nucleotide material;
drying the microencapsulated nucleotide material; and
grinding the dried microencapsulated nucleotide material to form the feed supplement.

9. The method of claim 9, wherein the microencapsulated nucleotide material is one of freeze-dried or spray-dried.

10. A feed formulation, comprising:
a nutrient base; and
a microparticulate nucleotide supplement, the microparticulate nucleotide supplement including a core material encapsulated within a protein coating material.

11. The feed formulation of claim 10, wherein the core material comprises at least one compound selected from the group consisting of nucleotides, uracil, oligonucleotides and combinations thereof.

12. The feed formulation of claim 11, wherein the core material further comprises a thickening agent.

13. The feed formulation of claim 10, wherein the protein coating material is selected from the group consisting of casein, gelatin, whey protein concentrate and combinations thereof.

14. The feed formulation of claim 10, wherein the microparticulate nucleotide supplement comprises about 20% to about 60% core material and about 30% to about 80% protein coating material.

15. The feed formulation of claim 10, wherein the microparticulate nucleotide supplement has a particle size in a range of from about 20 microns to about 150 microns.

16. A microencapsulated nucleotide supplement, comprising:
about 1% to about 50% of at least one nucleotide;
about 1% to about 30% of a thickening agent; and
about 30% to about 70% of a protein coating material.

17. The microencapsulated nucleotide supplement of claim 16, wherein the at least one nucleotide material is selected from the group consisting of AMP, CMP, GMP, UMP, IMP and Combinations thereof.

18. The microencapsulated nucleotide supplement of claim 16, wherein the protein coating material is selected from the group consisting of casein, gelatin, whey protein concentrate and combinations thereof.

19. The microencapsulated nucleotide supplement of claim 16, further comprising about 0.01% to about 50% of an immune system stimulating compound.

20. The microencapsulated nucleotide supplement of claim 16, further comprising about 0.01% to about 50% of a growth stimulating compound.
